(19) United States
(12) Patent Application Publication
(10) Pub. No.: US 2024/0165223 A1
(43) Pub. Date: May 23, 2024

(54) VACCINE COMPOSITION COMPRISING A LEISHMANIA HOST CELL EXPRESSING AT LEAST ONE PROTEIN OF THE FAMILY CORONAVIRIDAE

(71) Applicants: Universita' Degli Studi di Milano (90%), Milano (IT); Vismederi S.R.L. (10%)

(72) Inventors: Claudio Bandi, Pavia (IT); Sara Epis, Trescore Balneario (Bergamo) (IT); Ilaria Varotto Boccazzi, MIlano (IT); Gian Vincenzo Zuccotti, Senago (MIlano) (IT); Paolo Gabrieli, Milano (IT); Paolo Fiorina, Boston, MA (US); Louise Jane Gourlay, Milano (IT); Emanuele Montomoli, Siena (IT); Alessandro Manenti, Siena (IT)

(73) Assignees: Universita' Degli Studi di Milano (90%), Milano (IT); Vismederi S.R.L. (10%), (US)

(21) Appl. No.: 18/549,941

(22) PCT Filed: Feb. 23, 2022
(86) PCT No.: PCT/IB2022/051585

§ 371 Date: Sep. 11, 2023

(30) Foreign Application Priority Data
Feb. 23, 2021 (IT) 102021000004172

(51) Int. Cl. A61K 39/215; A61K 39/00; A61P 31/14; A61P 37/04; C12N 15/86

(52) U.S. Cl. CPC A61K 39/215; A61P 31/14; A61P 37/04; C12N 15/86; A61K 2039/523; A61K 2039/55505; A61K 2039/55511; A61K 2039/575; C12N 2770/20022; C12N 2770/20043; C12N 2770/20052; C12N 2770/20071

(57) ABSTRACT

The present invention relates to a vaccine composition comprising a host cell belonging to the genus Leishmania, wherein the host cell comprises a polynucleotide coding for at least one protein of a virus belonging to the family Coronaviridae. Furthermore, the invention relates to the medical and veterinary use of the vaccine composition and to a process for preparing the vaccine composition.

### TECHNICAL FIELD

The present invention relates to a vaccine composition comprising a host cell belonging to the genus Leishmania, wherein the host cell comprises a polynucleotide coding for at least one protein of a virus belonging to the family Coronaviridae. Furthermore, the invention relates to the medical and veterinary use of the vaccine composition and to a process for preparing the vaccine composition.

### PRIOR ART

Since the genetic sequence of the SARS-COV-2 virus was published on 11 Jan. 2020, scientists, industries and other organisations all over the world have worked together in order to develop safe, effective vaccines against COVID-19 as quickly as possible.

According to the overview of the World Health Organization (WHO), as of 22 Jan. 2021 there were 237 candidate vaccines undergoing development, 173 of which in a pre-clinical phase and 64 in a clinical phase (16 of the latter in phase 3). Some vaccines are produced using the same technology (or “platform”) as vaccines currently in use, others are produced using new approaches or approaches used in the development of vaccines against SARS and Ebola. The objective of all these vaccines is to produce an immune response with the aim of neutralising the virus and preventing the infection of cells. The main platforms used are the following: inactivated virus vaccines, live attenuated vaccines, recombinant protein vaccines, viral vector vaccines, DNA vaccines and RNA vaccines.

The vaccines and candidate vaccines proposed to date do not provide for an antigen delivery system aimed at dendritic cells (and other cells responsible for antigen presentation) and lymph nodes; the antigen is rather delivered indiscriminately to all the cells which are in some way exposed to the vaccine vehicle. This implies the possibility of limits in the generation of a long-term immunological memory (differentiation of long-lived plasma cells and memory B lymphocytes).

Furthermore, it cannot be ruled out that delivery of the antigen or of the vaccine vehicle to a wide range of cell types may have negative long-term effects.

The vaccines and candidate vaccines against SARS-COV-2 proposed to date have not been developed on the basis of an ability to reduce pro-inflammatory effects. The excessive inflammatory response at the site of the lymph nodes (e.g. with excessive production of TNF alpha) has been associated with an obstacle to the maturation of B lymphocytes, a reduced formation of germinal centres in lymph nodes, and thus a reduced formation of long-lived plasma cells, a drop in antibody concentrations, and poor differentiation of memory B lymphocytes. This results in a reduction in memory and antibody specificity.

For these reasons, there is a greatly felt need to develop a new vaccine against the SARS-COV-2 virus that is able to convey the antigen into the immune cells responsible for the presentation of antigens (professional APCs) and the transport thereof to secondary lymphoid organs (e.g. lymph nodes), while simultaneously reducing the inflammatory component.

### SUMMARY OF THE INVENTION

A first aspect of the present invention relates to a vaccine composition comprising a host cell belonging to the genus Leishmania; said host cell comprises a polynucleotide coding for at least one protein of a virus belonging to the family Coronaviridae or for at least one portion of said protein.

A second aspect of the present invention relates to the above-described vaccine composition for use as a medicament.

A third aspect of the present invention relates to the above-described vaccine composition for use in the prevention of a viral infection or a pathology caused by a virus belonging to the family Coronaviridae. In one embodiment the virus is selected from: SARS-COV, MERS-COV and SARS-COV-2. The virus is preferably SARS-COV-2.

A fourth aspect of the present invention relates to a host cell belonging to the genus Leishmania and coding for at least one protein of a virus belonging to the family Coronaviridae or for at least one portion of said protein.

A fifth aspect of the present invention relates to a process for preparing a vaccine composition; the process comprises the steps of:
a) introducing, into a host cell belonging to the genus Leishmania, a vector comprising a polynucleotide coding for at least one protein of a virus belonging to the family Coronaviridae or for at least one portion of said protein;
(b) selecting at least one host cell produced in step (a) that expresses said protein or portion of said protein; and
(c) suspending the at least one cell selected in step (b) with pharmaceutically acceptable salts/excipients.

A sixth aspect of the present invention relates to a method for preventing a viral infection or a pathology caused by a virus belonging to the family Coronaviridae. In one embodiment the virus is selected from: SARS-COV, MERS-COV and SARS-COV-2. The virus is preferably SARS-COV-2.

### BRIEF DESCRIPTION OF THE DRAWINGS

The present invention will be described in detail below and exemplified by way of non-limiting demonstration, also with the aid of the figures below.

FIG. 1 shows the plasmid used to engineer Leishmania tarentolae (A) and the gene fragment coding for the SARS-COV-2 spike protein inserted therein (B). A histidine six (H6) sequence was inserted downstream of the spike coding sequence;

FIG. 2 shows an evaluation of the production of the spike protein from a cell pellet by western blot. Samples Number 1-9: culture pellet; M: molecular weight (kDa) marker;

FIG. 3 shows Giemsa staining of human dendritic cells (A and B) and murine macrophages (C and D) following 4 hours of incubation with the protozoan L. tarentolae engineered according to the present invention for the production of the spike protein (100× magnification);

FIG. 4 shows the presence of the spike protein within the dendritic cells (DCs) after coincubation with L. tarentolae modified for the production of this protein, determined by means of immunofluorescence assays;

FIG. 5 shows the expression of markers of human dendritic cell activation following incubation with Lt-spike.

### DEFINITIONS

In the context of the present invention, the term “spike” means a glycoprotein structure present as a protuberance on the outside of the viral envelope, the lipid bilayer that constitutes the outer wrapping of some viruses. These protuberances bind to several receptors of the host cell and are essential both for the specificity of the host and for viral infectivity.

In the context of the present invention, the term Coronaviridae (abbreviated as “CoV”) means a large family of respiratory viruses that can cause mild to moderate illnesses, which range from the common cold to respiratory syndromes such as MERS (Middle East respiratory syndrome) and SARS (severe acute respiratory syndrome), in addition to other viruses responsible for pathologies, not only respiratory ones, in domestic and wild animals.

In the context of the present invention, the term “infection” means a process characterised by the penetration and multiplication of viruses in living tissue. The concept of infection is not identifiable with that of infectious disease or pathology, as there exist cases of infection without any morbid phenomenon, i.e. asymptomatic individuals who are carriers of the pathogen.

### DETAILED DESCRIPTION OF PREFERRED EMBODIMENTS OF THE INVENTION

A first aspect of the present invention relates to a vaccine composition comprising a host cell belonging to the genus Leishmania; said host cell comprises a polynucleotide coding for at least one protein of a virus belonging to the family Coronaviridae or for at least one portion of said protein.

In one embodiment, the polynucleotide coding for said viral protein is selected from DNA and RNA; it is preferably DNA.

In one embodiment, the polynucleotide comprises a nucleotide sequence that is substantially identical to SEQ ID NO.1 or SEQ ID NO.2 (Table 1). For example, the polynucleotide comprises a nucleotide sequence that is at least 50%, 55%, 60%, 65%, 70%, 75%, 80%, 85%, 90%, 91%, 92%, 93%, 94%, 95%, 96%, 97%, 98% or 99% identical or more to SEQ ID NO. 1 or SEQ ID NO.2.

The polynucleotide preferably comprises a nucleotide sequence that is at least 80% identical, more preferably at least 90% identical to SEQ ID NO. 1 or SEQ ID NO.2.

In one embodiment, the polynucleotide consists in a nucleotide sequence that is substantially identical to SEQ ID NO.1 or SEQ ID NO.2. For example, the polynucleotide consists in a nucleotide sequence that is at least 50%, 55%, 60%, 65%, 70%, 75%, 80%, 85%, 90%, 91%, 92%, 93%, 94%, 95%, 96%, 97%, 98% or 99% identical or more to SEQ ID NO. 1 or SEQ ID NO.2.

The polynucleotide preferably consists in a nucleotide sequence that is homologous or at least 80% identical, more preferably at least 90% identical to SEQ ID NO. 1 or SEQ ID NO.2.

The at least one protein is preferably a protein, preferably a spike glycoprotein, or at least a portion thereof, of a virus that is a member of the family Coronaviridae, selected from: SARS-COV, MERS-COV and SARS-CoV-2. In a preferred embodiment of the invention, the at least one protein is a protein, preferably a spike glycoprotein, of the virus SARS-COV-2 or at least a portion thereof.

In one embodiment, the at least one protein comprises an amino acid sequence that is substantially identical to SEQ ID NO.3 or SEQ ID NO.4. For example, the at least one protein comprises an amino acid sequence that is at least 50%, 55%, 60%, 65%, 70%, 75%, 80%, 85%, 90%, 91%, 92%, 93%, 94%, 95%, 96%, 97%, 98% or 99% identical or more to SEQ ID NO. 3 or SEQ ID NO.4.

In a preferred embodiment, the at least one protein comprises an amino acid sequence that is at least 80% identical, more preferably at least 90% identical to SEQ ID NO. 3 or SEQ ID NO.4.

In one embodiment, the at least one protein consists in an amino acid sequence that is substantially identical to SEQ ID NO. 3 or SEQ ID NO.4. For example, the at least one protein consists in an amino acid sequence that is at least 50%, 55%, 60%, 65%, 70%, 75%, 80%, 85%, 90%, 91%, 92%, 93%, 94%, 95%, 96%, 97%, 98% or 99% identical or more to SEQ ID NO. 3 or SEQ ID NO.4.

In a preferred embodiment, the at least one protein consists in an amino acid sequence that is at least 80% identical, more preferably at least 90% identical to SEQ ID NO. 3 or SEQ ID NO.4.

Said at least one protein of a virus is preferably the spike protein, or at least a portion thereof, of a virus that is a member of the family Coronaviridae.

Said at least one protein of a virus is preferably the spike protein, or at least a portion thereof, of SARS-COV-2.

In one embodiment of the invention, the at least one protein comprises modifications to the N-terminal and/or C-terminal region. Said modifications are preferably selected from deletions, additions, alterations of amino acids and combinations thereof. Alternatively, said at least one protein can be modified, preferably in its primary structure, or by acetylation, carboxylation, phosphorylation and combinations thereof.

In one embodiment of the invention, the host cell that expresses the above-described polynucleotide belongs to a specie of Leishmania selected from: L. tarentolae, L. infantum. L. donovani, and L. major. The host cell preferably belongs to the species L. tarentolae.

In one embodiment, the composition further comprises at least one protein, wherein the at least one protein is a spike glycoprotein, or at least a portion thereof, of a virus that is a member of the family Coronaviridae and selected from: SARS-COV, MERS-COV and SARS-COV-2. In a preferred embodiment of the invention, the at least one protein is a protein, preferably a spike glycoprotein, of the virus SARS-COV-2 or at least a portion thereof, as described above in detail. In other words, the composition also comprises, in addition to the host cell belonging to the genus Leishmania as described above, a protein, preferably a spike glycoprotein of a virus that is a member of the family Coronaviridae, as described above in detail.

In one embodiment, the above-described polynucleotide is comprised in a vector. In one embodiment, the vector comprising the above-described polynucleotide is selected from: viral vector, plasmid, viral particles and phage.

The vector is preferably a plasmid vector.

In one embodiment, the vector, preferably the plasmid, comprises at least one gene capable of imparting resistance to at least one antibiotic.

The plasmid is preferably engineered so as to favour the constitutive and/or inducible cytosolic and/or secretory expression of the at least one protein or a part thereof. In one embodiment, the polynucleotide is inserted into the vector, preferably into the plasmid, under the control of a promoter, for example the Pr-Leishmania ribosomal promoter.

In one embodiment, the sequence inserted into the engineered vector/plasmid is incorporated into the nuclear genome of the host cell of Leishmania.

The composition of the present invention can preferably further comprise at least one pharmacologically acceptable excipient, i.e. a compound, acceptable for pharmaceutical use, that is useful in the preparation of the composition and is generally biologically safe and nontoxic. Furthermore, the composition of the present invention can further comprise at least one adjuvant, i.e. a product aimed at enhancing the immune response against the viral protein, administered as envisaged by the present invention.

In one embodiment, the composition is formulated for parenteral administration, preferably as a solution, suspension, sterile emulsion, or powder to be resuspended prior to use.

In one embodiment, the composition is formulated for enteral administration, preferably as pills, capsules, tablets, granular powder, hard-shelled capsules, orally dissolving granules, sachets or lozenges.

In one embodiment, the composition is formulated for administration by inhalation.

A second aspect of the present invention relates to the vaccine composition described in detail above for use as a medicament.

The Applicant has in fact demonstrated that the vaccine composition of the present invention is capable of conveying the antigen into the immune cells responsible for antigen presentation (professional APCs), and in particular into dendritic cells, and thus probably the transport thereof to secondary lymphoid organs (e.g. lymph nodes). Furthermore, it is expected that the vaccine composition of the present invention will enable the lymph node response to be directed so as to favour the formation of germinal centres, antibody isotype switching and the differentiation of memory B cells and of long-lived plasma cells, with the aim of inhibiting a modulation of the immunological memory in a pro-inflammatory direction (Th1 and M1).

In fact, as demonstrated in the example, the host cell belonging to the genus Leishmania and contained in the vaccine composition described herein, is capable of being phagocytised by dendritic cells and cells of the macrophage line, thereby activating the immune system. In other words, the host cell belonging to the genus Leishmania is internalised both by macrophages and by dendritic cells. Furthermore, the host cell belonging to the genus Leishmania is capable of inducing a moderate production of cytokines, such as, for example, TNF, IL-12, IL-2, in dendritic cells incubated with said host cell. Finally, sera of SARS-COV-2-positive patients, that is, sera in which specific antibodies against the SARS-COV-2 virus were present, were capable of recognising the viral protein expressed by the host cell belonging to the genus Leishmania. In other words, the at least one viral protein expressed by the host cell contained in the vaccine composition is recognised by the specific antibodies present in the serum of SARS-COV-2-positive patients.

Therefore, a third aspect of the present invention relates to the composition described above in detail for use in the prevention of a viral infection or a pathology caused by a virus belonging to the family Coronaviridae. In one embodiment the virus is selected from: SARS-COV, MERS-COV and SARS-COV-2. The virus is preferably SARS-COV-2.

In one embodiment, the pathology caused by a virus of the family Coronaviridae is an infectious disease, an acute respiratory pathology, or a pathology of a systemic type, or an asymptomatic or oligosymptomatic infection, preferably caused by the virus SARS-COV-2 and known as COVID-19.

In one embodiment, for the above-described medical or veterinary uses, the vaccine composition is administered in a single dose.

In an alternative embodiment, for the above-described medical or veterinary uses, the vaccine composition is administered in two separate doses; the second dose is preferably administered at least 20 days after the first dose, more preferably at least 25 days afterwards.

In an alternative embodiment, for the above-described medical or veterinary uses, the vaccine composition is administered in more than two separate doses.

In a further embodiment, the vaccine composition, comprising a Leishmania modified for the expression of at least one protein (or portion of protein) of a virus of the family Coronaviridae is administered in association or in combination with at least one purified protein, wherein the at least one protein is preferably a spike glycoprotein, or at least a portion thereof, of a virus that is a member of the family Coronaviridae selected from: SARS-COV, MERS-COV and SARS-COV-2. In a preferred embodiment of the invention, the at least one protein is a protein, preferably a spike glycoprotein, of the virus SARS-COV-2 or at least a portion thereof as described above in detail.

TABLE 1

| | | |
|---|---|---|
| SEQ ID | GTTAATCTTACAACCAGAACTCAAT | Sequence |
| NO: 1 | TACCCCCTGCATACACTAATTCTTT | coding for |
| | CACACGTGGTGTTTATTACCCTGAC | SARS-CoV-2 |
| | AAAGTTTTCAGATCCTCAGTTTTAC | spike |
| | ATTCAACTCAGGACTTGTTCTTACC | protein |
| | TTTCTTTTCCAATGTTACTTGGTTC | |
| | CATGCTATACATGTCTCTGGGACCA | |
| | ATGGTACTAAGAGGTTTGATAACCC | |
| | TGTCCTACCATTTAATGATGGTGTT | |
| | TATTTTGCTTCCACTGAGAAGTCTA | |
| | ACATAATAAGAGGCTGGATTTTTGG | |
| | TACTACTTTAGATTCGAAGACCCAG | |
| | TCCCTACTTATTGTTAATAACGCTA | |
| | CTAATGTTGTTATTAAAGTCTGTGA | |
| | ATTTCAATTTTGTAATGATCCATTT | |
| | TTGGGTGTTTATTACCACAAAAACA | |
| | ACAAAAGTTGGATGGAAAGTGAGTT | |
| | CAGAGTTTATTCTAGTGCGAATAAT | |
| | TGCACTTTTGAATATGTCTCTCAGC | |
| | CTTTTCTTATGGACCTTGAAGGAAA | |
| | ACAGGGTAATTTCAAAAATCTTAGG | |
| | GAATTTGTGTTTAAGAATATTGATG | |
| | GTTATTTTAAAATATATTCTAAGCA | |
| | CACGCCTATTAATTTAGTGCGTGAT | |
| | CTCCCTCAGGGTTTTTCGGCTTTAG | |
| | AACCATTGGTAGATTTGCCAATAGG | |
| | TATTAACATCACTAGGTTTCAAACT | |
| | TTACTTGCTTTACATAGAAGTTATT | |
| | TGACTCCTGGTGATTCTTCTTCAGG | |
| | TTGGACAGCTGGTGCTGCAGCTTAT | |
| | TATGTGGGTTATCTTCAACCTAGGA | |
| | CTTTTCTATTAAAATATAATGAAAA | |
| | TGGAACCATTACAGATGCTGTAGAC | |
| | TGTGCACTTGACCCTCTCTCAGAAA | |
| | CAAAGTGTACGTTGAAATCCTTCAC | |
| | TGTAGAAAAAGGAATCTATCAAACT | |
| | TCTAACTTTAGAGTCCAACCAACAG | |
| | AATCTATTGTTAGATTTCCTAATAT | |
| | TACAAACTTGTGCCCTTTTGGTGAA | |
| | GTTTTTAACGCCACCAGATTTGCAT | |
| | CTGTTTATGCTTGGAACAGGAAGAG | |
| | AATCAGCAACTGTGTTGCTGATTAT | |
| | TCTGTCCTATATAATTCCGCATCAT | |
| | TTTCCACTTTTAAGTGTTATGGAGT | |
| | GTCTCCTACTAAATTAAATGATCTC | |
| | TGCTTTACTAATGTCTATGCAGATT | |
| | CATTTGTAATTAGAGGTGATGAAGT | |
| | CAGACAAATCGCTCCAGGGCAAACT | |
| | GGAAAGATTGCTGATTATAATTATA | |
| | AATTACCAGATGATTTTACAGGCTG | |
| | CGTTATAGCTTGGAATTCTAACAAT | |
| | CTTGATTCTAAGGTTGGTGGTAATT | |
| | ATAATTACCTGTATAGATTGTTTAG | |
| | GAAGTCTAATCTCAAACCTTTTGAG | |
| | AGAGATATTTCAACTGAAATCTATC | |
| | AGGCCGGTAGCACACCTTGTAATGG | |
| | TGTTGAAGGTTTTAATTGTTACTTT | |
| | CCTTTACAATCATATGGTTTCCAAC | |
| | CCACTAATGGTGTTGGTTACCAACC | |
| | ATACAGAGTAGTAGTACTTTCTTTT | |
| | GAACTTCTACATGCACCAGCAACTG | |
| | TTTGTGGACCTAAAAAGTCTACTAA | |
| | TTTGGTTAAAAACAAATGTGTCAAT | |
| | TTCAACTTCAATGGTTTAACAGGCA | |
| | CAGGTGTTCTTACTGAGTCTAACAA | |
| | AAAGTTTCTGCCTTTCCAACAATTT | |
| | GGCAGAGACATTGCTGACACTACTG | |
| | ATGCTGTCCGTGATCCACAGACACT | |
| | TGAGATTCTTGACATTACACCATGT | |
| | TCTTTTGGTGGTGTCAGTGTTATAA | |
| | CACCAGGAACAAATACTTCTAACCA | |
| | GGTTGCTGTTCTTTATCAGGGTGTT | |
| | AACTGCACAGAAGTCCCTGTTGCTA | |
| | TTCATGCAGATCAACTTACTCCTAC | |
| | TTGGCGTGTTTATTCTACAGGTTCT | |
| | AATGTTTTTCAAACACGTGCAGGCT | |
| | GTTTAATAGGGGCTGAACATGTCAA | |
| | CAACTCATATGAGTGTGACATACCC | |
| | ATTGGTGCAGGTATATGCGCTAGTT | |
| | ATCAGACTCAGACTAATTCTCCTGG | |
| | TTCTGCATCTAGTGTAGCTAGTCAA | |
| | TCCATCATTGCCTACACTATGTCAC | |
| | TTGGTGCAGAAAATTCAGTTGCTTA | |
| | CTCTAATAACTCTATTGCCATACCC | |
| | ACAAATTTTACTATTAGTGTTACCA | |
| | CAGAAATTCTACCAGTGTCTATGAC | |
| | CAAGACATCAGTAGATTGTACAATG | |
| | TACATTTGTGGTGATTCAACTGAAT | |
| | GCAGCAATCTTTTGTTGCAATATGG | |
| | CAGTTTTTGTACACAATTAAACCGT | |
| | GCTTTAACTGGAATAGCTGTTGAAC | |
| | AAGACAAAAACACCCAAGAAGTTTT | |
| | TGCACAAGTCAAACAAATTTACAAA | |
| | ACACCACCAATTAAAGATTTTGGTG | |
| | GTTTTAATTTTTCACAAATATTACC | |
| | AGATCCATCAAAACCAAGCAAGAGG | |
| | TCATTTATTGAAGATCTACTTTTCA | |
| | ACAAAGTGACACTTGCAGATGCTGG | |
| | CTTCATCAAACAATATGGTGATTGC | |
| | CTTGGTGATATTGCTGCTAGAGACC | |
| | TCATTTGTGCACAAAAGTTTAACGG | |
| | CCTTACTGTTTTGCCACCTTTGCTC | |
| | ACAGATGAAATGATTGCTCAATACA | |
| | CTTCTGCACTGTTAGCGGGTACAAT | |
| | CACTTCTGGTTGGACCTTTGGTGCA | |
| | GGTGCTGCATTACAAATACCATTTG | |
| | CTATGCAAATGGCTTATAGGTTTAA | |
| | TGGTATTGGAGTTACACAGAATGTT | |
| | CTCTATGAGAACCAAAAATTGATTG | |
| | CCAACCAATTTAATAGTGCTATTGG | |
| | CAAAATTCAAGACTCACTTTCTTCC | |
| | ACAGCAAGTGCACTTGGAAAACTTC | |
| | AAGATGTGGTCAACCAAAATGCACA | |
| | AGCTTTAAACACGCTTGTTAAACAA | |
| | CTTAGCTCCAATTTTGGTGCAATTT | |
| | CAAGTGTTTTAAATGATATCCTTTC | |
| | ACGTCTTGACAAAGTTGAGGCTGAA | |
| | GTGCAAATTGATAGGTTGATCACAG | |
| | GCAGACTTCAAAGTTTGCAGACATA | |
| | TGTGACTCAACAATTAATTAGAGCT | |
| | GCAGAAATCAGAGCTTCTGCTAATC | |
| | TTGCTGCTACTAAAATGTCAGAGTG | |
| | TGTACTTGGACAATCAAAAAGAGTT | |
| | GATTTTTGTGGAAAGGGCTATCATC | |
| | TTATGTCCTTCCCTCAGTCAGCACC | |
| | TCATGGTGTAGTCTTCTTGCATGTG | |
| | ACTTATGTCCCTGCACAAGAAAAGA | |
| | ACTTCACAACTGCTCCTGCCATTTG | |
| | TCATGATGGAAAAGCACACTTTCCT | |
| | CGTGAAGGTGTCTTTGTTTCAAATG | |
| | GCACACACTGGTTTGTAACACAAAG | |
| | GAATTTTTATGAACCACAAATCATT | |
| | ACTACAGACAACACATTTGTGTCTG | |
| | GTAACTGTGATGTTGTAATAGGAAT | |
| | TGTCAACAACACAGTTTATGATCCT | |
| | TTGCAACCTGAATTAGACTCATTCA | |
| | AGGAGGAGTTAGATAAATATTTTAA | |
| | GAATCATACATCACCAGATGTTGAT | |
| | TTAGGTGACATCTCTGGCATTAATG | |
| | CTTCAGTTGTAAACATTCAAAAAGA | |
| | AATTGACCGCCTCAATGAGGTTGCC | |
| | AAGAATTTAAATGAATCTCTCATCG | |
| | ATCTCCAAGAACTTGGAAAGTATGA | |
| | GCAGTATATAAAATGGCCATGGTAC | |
| | ATTTGGCTAGGTTTTATAGCTGGCT | |
| | TGATTGCCATAGTAATGGTGACAAT | |
| | TATGCTTTGCTGTATGACCAGTTGC | |
| | TGTAGTTGTCTCAAGGGCTGTTGTT | |
| | CTTGTGGATCCTGCTGCAAA | |
| SEQ ID | ATGGCCCGTGTGCAGCCAACTGAGA | Sequence |
| NO: 2 | GCATTGTGCGCTTCCCGAACATCAC | coding for |
| | GAACCTGTGCCCATTCGGCGAGGTG | SARS-CoV-2 |
| | TTCAACGCGACACGTTTCGCGAGCG | RBD-SD1 |
| | TGTACGCGTGGAACCGCAAGCGCAT | |
| | TAGCAACTGCGTGGCGGACTACAGC | |
| | GTGCTGTACAACAGCGCGAGCTTCA | |
| | GCACGTTCAAGTGCTACGGCGTGTC | |
| | GCCGACGAAGCTGAACGACCTGTGC | |
| | TTCACGAACGTGTACGCCGACAGCT | |
| | TTGTGATCCGTGGCGACGAGGTCCG | |
| | CCAGATTGCACCAGGCCAGACAGGC | |
| | AAGATCGCCGACTACAACTACAAGC | |
| | TGCCGGACGACTTTACGGGCTGCGT | |
| | GATCGCATGGAACAGCAACAACCTG | |
| | GACAGCAAGGTCGGCGGCAACTACA | |
| | ACTACCTGTACCGCCTGTTCCGCAA | |
| | GAGCAACCTGAAGCCGTTTGAGCGC | |
| | GACATCAGCACCGAGATCTACCAGG | |
| | CAGGTTCTACGCCATGCAACGGCGT | |
| | CGAGGGCTTCAACTGCTACTTTCCG | |
| | CTGCAGTCGTACGGCTTCCAGCCGA | |
| | CAAACGGTGTCGGCTACCAGCCATA | |
| | CCGTGTGGTGGTGCTGTCTTTCGAG | |
| | CTGCTGCACGCACCAGCAACAGTGT | |
| | GCGGTCCAAAGAAGTCCACCAACCT | |
| | GGTCAAGAACAAGTGCGTGAACTTC | |
| | AACTTCAACGGCCTGACCGGCACGG | |
| | GCGTGCTGACAGAGAGCAACAAGAA | |
| | GTTCCTGCCGTTCCAGCAGTTCGGC | |
| | CGCGACATTGCAGACACGACAGATG | |
| | CAGTGCGCGACCCGCAGACGCTTGA | |
| | GATCCTGGACATTACACCGTGCAGC | |
| SEQ ID | VNLTTRTQLPPAYTNSFTRGVYYPD | Amino acid |
| NO: 3 | KVFRSSVLHSTQDLFLPFFSNVTWF | sequence of |
| | HAIHVSGTNGTKRFDNPVLPFNDGV | SARS-CoV-2 |
| | YFASTEKSNIIRGWIFGTTLDSKTQ | spike |
| | SLLIVNNATNVVIKVCEFQFCNDPF | protein |
| | LGVYYHKNNKSWMESEFRVYSSANN | |
| | CTFEYVSQPFLMDLEGKQGNFKNLR | |
| | EFVFKNIDGYFKIYSKHTPINLVRD | |
| | LPQGFSALEPLVDLPIGINITRFQT | |
| | LLALHRSYLTPGDSSSGWTAGAAAY | |
| | YVGYLQPRTFLLKYNENGTITDAVD | |
| | CALDPLSETKCTLKSFTVEKGIYQT | |
| | SNFRVQPTESIVRFPNITNLCPFGE | |
| | VFNATRFASVYAWNRKRISNCVADY | |
| | SVLYNSASFSTFKCYGVSPTKLNDL | |
| | CFTNVYADSFVIRGDEVRQIAPGQT | |
| | GKIADYNYKLPDDFTGCVIAWNSNN | |
| | LDSKVGGNYNYLYRLFRKSNLKPFE | |
| | RDISTEIYQAGSTPCNGVEGFNCYF | |
| | PLQSYGFQPTNGVGYQPYRVVVLSF | |
| | ELLHAPATVCGPKKSTNLVKNKCVN | |
| | FNFNGLTGTGVLTESNKKFLPFQQF | |
| | GRDIADTTDAVRDPQTLEILDITPC | |
| | SFGGVSVITPGTNTSNQVAVLYQGV | |
| | NCTEVPVAIHADQLTPTWRVYSTGS | |
| | NVFQTRAGCLIGAEHVNNSYECDIP | |
| | IGAGICASYQTQTNSPGSASSVASQ | |
| | SIIAYTMSLGAENSVAYSNNSIAIP | |
| | TNFTISVTTEILPVSMTKTSVDCTM | |
| | YICGDSTECSNLLLQYGSFCTQLNR | |
| | ALTGIAVEQDKNTQEVFAQVKQIYK | |
| | TPPIKDFGGFNFSQILPDPSKPSKR | |
| | SFIEDLLFNKVTLADAGFIKQYGDC | |
| | LGDIAARDLICAQKFNGLTVLPPLL | |
| | TDEMIAQYTSALLAGTITSGWTFGA | |
| | GAALQIPFAMQMAYRFNGIGVTQNV | |
| | LYENQKLIANQFNSAIGKIQDSLSS | |
| | TASALGKLQDVVNQNAQALNTLVKQ | |
| | LSSNFGAISSVLNDILSRLDKVEAE | |
| | VQIDRLITGRLQSLQTYVTQQLIRA | |
| | AEIRASANLAATKMSECVLGQSKRV | |
| | DFCGKGYHLMSFPQSAPHGVVFLHV | |
| | TYVPAQEKNFTTAPAICHDGKAHFP | |
| | REGVFVSNGTHWFVTQRNFYEPQII | |
| | TTDNTFVSGNCDVVIGIVNNTVYDP | |
| | LQPELDSFKEELDKYFKNHTSPDVD | |
| | LGDISGINASVVNIQKEIDRLNEVA | |
| | KNLNESLIDLQELGKYEQYIKWPWY | |
| | IWLGFIAGLIAIVMVTIMLCCMTSC | |
| | CSCLKGCCSCGSCCKGTHHHHHH | |
| SEQ ID | MARVQPTESIVRFPNITNLCPFGEV | Amino acid |
| NO: 4 | FNATRFASVYAWNRKRISNCVADYS | sequence of |
| | VLYNSASFSTFKCYGVSPTKLNDLC | SARS-CoV-2 |
| | FTNVYADSFVIRGDEVRQIAPGQTG | RBD-SD1 |
| | KIADYNYKLPDDFTGCVIAWNSNNL | |
| | DSKVGGNYNYLYRLFRKSNLKPFER | |
| | DISTEIYQAGSTPCNGVEGFNCYFP | |
| | LQSYGFQPTNGVGYQPYRVVVLSFE | |
| | LLHAPATVCGPKKSTNLVKNKCVNF | |
| | NFNGLTGTGVLTESNKKFLPFQQFG | |
| | RDIADTTDAVRDPQTLEILDITPCS | |

A fourth aspect of the present invention relates to a host cell belonging to the genus Leishmania coding for at least one protein of a virus belonging to the family Coronaviridae or for at least one portion of said protein.

In one embodiment, the polynucleotide comprises or consists in a nucleotide sequence that is substantially identical to SEQ ID NO.1 as described above in detail.

In one embodiment, the polynucleotide comprises or consists in a nucleotide sequence that is substantially identical to SEQ ID NO.2 as described above in detail.

In a preferred embodiment of the invention, the at least one protein of a virus belonging to the family Coronaviridae is a spike glycoprotein or at least a portion thereof. In one embodiment, the at least one protein comprises or consists in an amino acid sequence that is substantially identical to SEQ ID NO.3 as described above in detail. In a further embodiment, the at least one protein comprises or consists in an amino acid sequence that is substantially identical to SEQ ID NO.4 as described above in detail.

A fifth aspect of the present invention relates to a process for preparing a vaccine composition; the process comprises the steps of:
a) introducing, into a host cell belonging to the genus Leishmania, a vector comprising a polynucleotide coding for at least one protein of a virus belonging to the family Coronaviridae or for at least one portion of said protein;
(b) selecting at least one host cell produced in step (a) that expresses said protein or portion of said protein; and
(c) suspending the at least one cell selected in step (b) with pharmaceutically acceptable salts/excipients.

In one embodiment, the polynucleotide comprises or consists in a nucleotide sequence that is substantially identical to SEQ ID NO.1 or SEQ ID NO.2 as described above in detail.

In one embodiment, the vector comprising the polynucleotide described above in detail is inserted into the cell of Leishmania using the techniques known to the skilled person. For example, the introduction of the vector takes place by electroporation, or by treating the cell of Leishmania with lithium acetate. In a preferred embodiment, the vector comprising the above-described polynucleotide is inserted into the cell of Leishmania by electroporation.

In one embodiment, step (b) comprises a sub-step (b1) of clonal selection and a sub-step (b2) of verifying the selected clones.

Sub-step (b1) preferably comprises the selection of Leishmania clones that express the viral protein or portion of said viral protein through the use of culture media comprising at least one antibiotic. In fact, the vector, preferably the plasmid, preferably comprises at least one gene capable of imparting to the Leishmania resistance to at least one antibiotic.

Sub-step (b2) preferably comprises verifying the actual integration of the vector into the genome of the Leishmania clones selected in step (b1). Said sub-step (b2) comprises checking for the presence of the vector and/or of the polynucleotide inserted in step (a) using molecular biology techniques known to the person skilled in the art, for example using PCR techniques and/or electrophoresis techniques and/or immunoblotting.

In a preferred embodiment of the invention, the at least one protein of a virus belonging to the family Coronaviridae is a spike glycoprotein or at least a portion thereof. In one embodiment, the at least one protein comprises or consists in an amino acid sequence that is substantially identical to SEQ ID NO.3 or SEQ ID NO.4 as described above in detail.

A sixth aspect of the present invention relates to a method for preventing a viral infection or a pathology caused by a virus belonging to the family Coronaviridae. In one embodiment the virus is selected from: SARS-COV, MERS-COV and SARS-COV-2. The virus is preferably SARS-COV-2.

In one embodiment, the pathology caused by a virus of the family Coronaviridae is an infectious disease, an acute respiratory pathology, or a pathology of a systemic type, or an asymptomatic or oligosymptomatic infection, preferably caused by the virus SARS-COV-2 and known as COVID-19.

The method comprises at least one step of administering the vaccine composition described above in detail to an individual.

In one embodiment, the vaccine composition is administered in a single dose.

In an alternative embodiment, the vaccine composition is administered in two separate doses; the second dose is preferably administered at least 20 days after the first dose, more preferably at least 25 days afterwards.

In an alternative embodiment, the vaccine composition is administered in more than two separate doses.

### Example

1. Engineering of the Protozoan Leishmania tarentolae for Expression of the SARS-COV-2 Spike Protein.

A strain of Leishmania tarentolae was generated for cytosolic expression of the spike protein, hereinafter indicated as LeCoVax2. The sequence of the spike protein was derived from the genome of “severe acute respiratory syndrome coronavirus 2 isolate Wuhan-Hu-1”, number MN908947. 3720 nucleotides were selected for a total of 1240 amino acids. The spike gene sequence was modified by introducing a substitution of the amino acids “GSAS” at the furin cleavage site (residues 682-685). The spike gene sequence was synthesised by inserting the C-terminal tail GTHHHHHH. The expression vector pLEXSY-sat2.1 (Jena Bioscience) was selected for cytosolic expression of the target protein.

The target gene was inserted into the expression cassette under the control of the Pr-Leishmania ribosomal promoter. The strain of L. tarentolae thus engineered was cultured at 26° C. under aerobiosis in BHI liquid medium supplemented with hemin, penicillin-streptomycin and nourseothricin for selection.

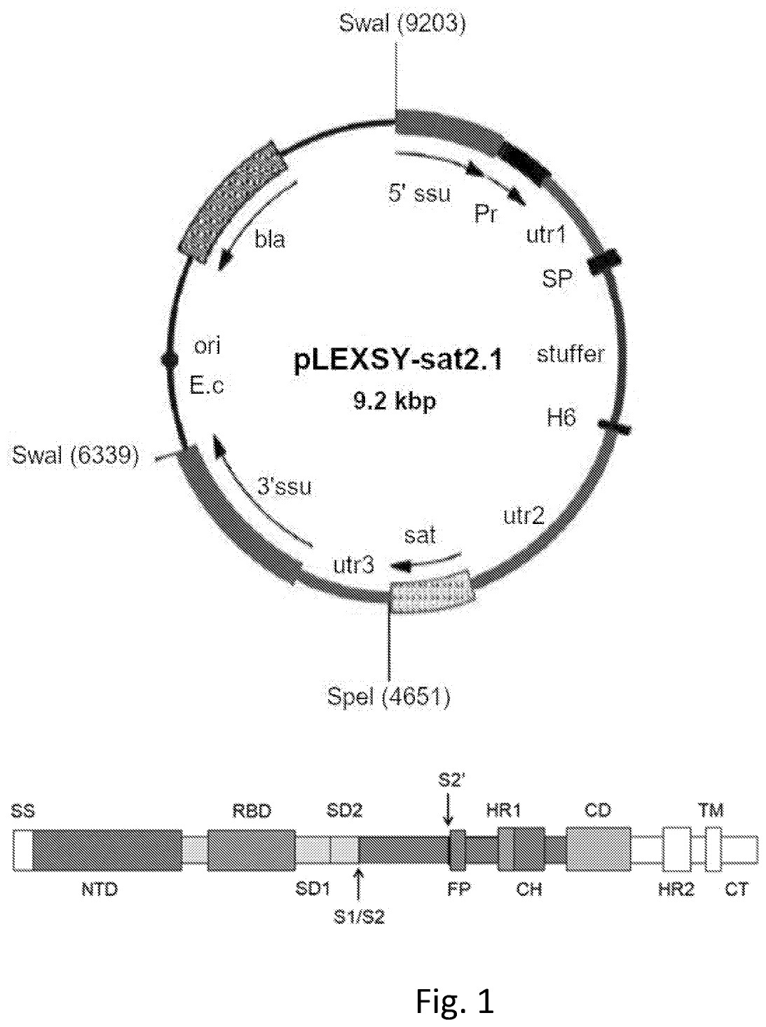

FIG. 1 shows information relating to the plasmid used and to the insert. The construct was designed so as to obtain production of the protein in cytosolic form

2. Expression of the Lt-Spike Antigen Produced in L. tarentolae

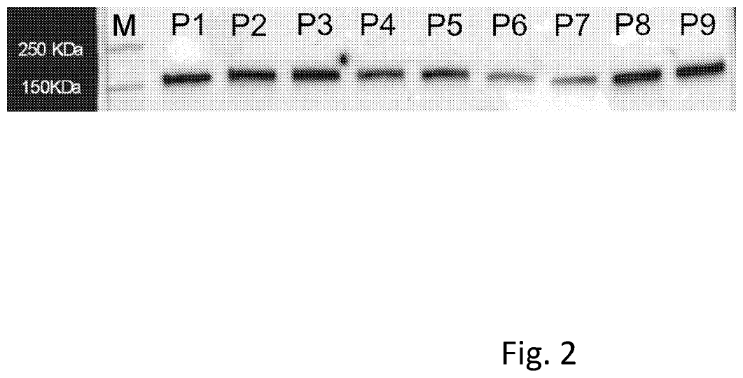

The expression of the Lt-Spike protein was evaluated by Western Blot analysis starting from the cell pellet, using the SARS Coronavirus Spike Protein polyclonal antibody (1:3000). As shown in FIG. 2, a band weighing approximately 180 kDa is present in all of the pellet samples analysed.

3. Internalisation of Spike-Producing Leishmania tarentolae by Cells Presenting the Antigen

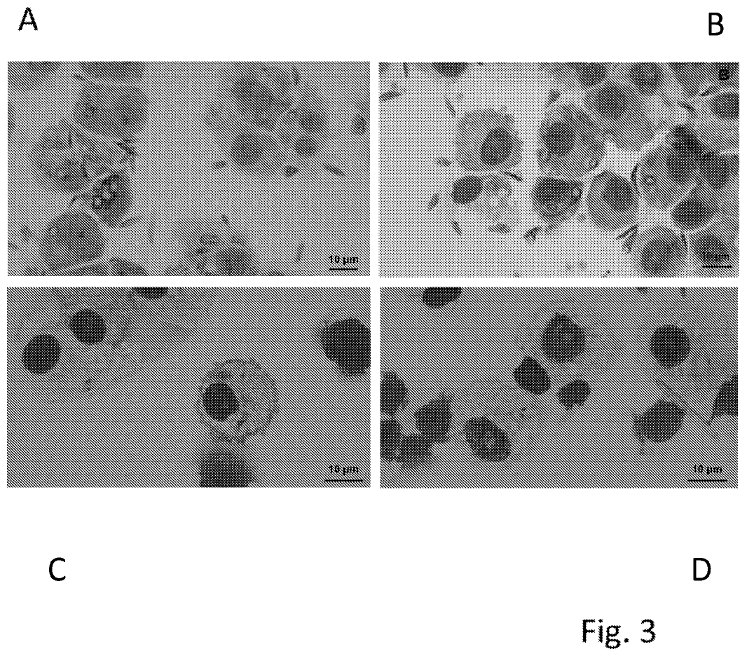

One of the prerequisites for the development of LeCoVax2 is the ability of Leishmania to be actively phagocytised by cells of the macrophage monocyte system and by dendritic cells. The ability of the protozoan Leishmania tarentolae to be phagocytised both by murine macrophages and by dendritic cells of human origin was tested in in vitro experiments. The cells were incubated with the protozoa at two different macrophage/dendritic cell: Leishmania ratios (1:5 or 1:10) for 4 h. At the end of the incubation period, the cells were cytocentrifuged and stained with Giemsa stain in order to observe, under an optical microscope, the actual internalisation of the parasites within the antigen-presenting cells and calculate the infection rate thereof (percentage of cells with at least one parasite) (FIG. 3). As shown in the panels in FIG. 3, after 4 hours L. tarentolae is internalised both by dendritic cells (FIGS. 3A and B) and by macrophages (FIGS. 3 C and D); it is possible, in fact, to observe numerous parasites, which in many cases appear to be degraded, inside the cells. In particular, this phenomenon appears to be particularly accelerated in macrophages compared to dendritic cells (FIGS. 3 C and D). The rate of infection of the macrophages incubated with L. tarentolae-Spike 1:5 and 1:10 is 16% and 51% respectively.

4. Recognition of the Lt-RBD-SD1 Antigen Produced by L. tarentolae by Sera of SARS-COV-2-Positive Subjects

Using a standard method (western blot) it was verified that the sera of SARS-COV-2-positive patients recognise the purified Lt-RBD-SD1 antigen. Furthermore, the same antigen was examined by ELISA and demonstrated to be specifically recognised by sera of patients infected by SARS-COV-2, but not recognised by sera of subjects who were negative for the virus.

5. Immunofluorescence on Dendritic Cells Infected with Lt Spike and Lt_WT

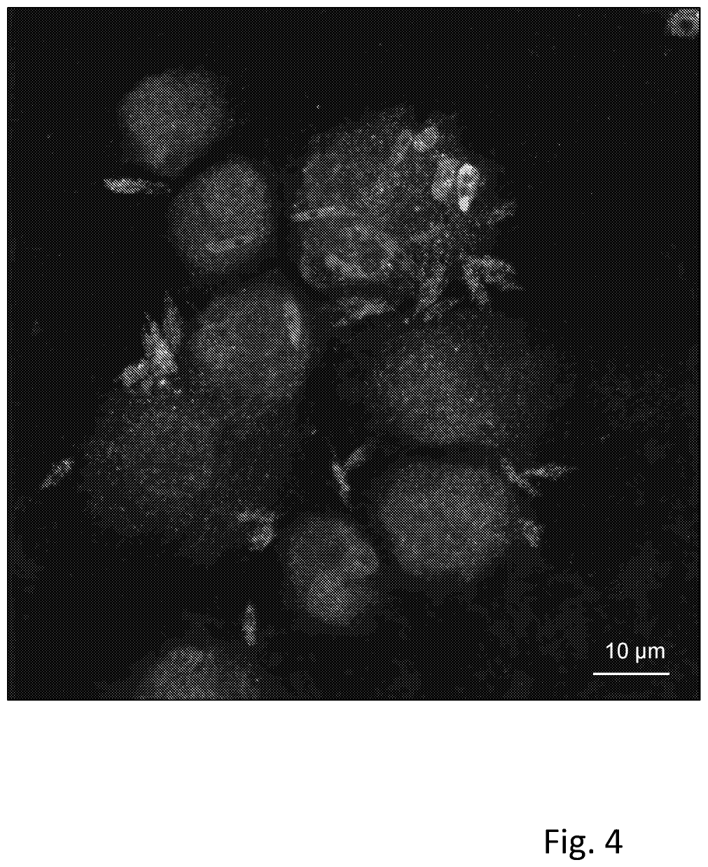

The assays were conducted on dendritic cells (DCs). The DCs were differentiated from monocytes from blood of healthy donors. The dendritic cells were subsequently exposed to wild-type L. tarentolae (Lt_WT) and Lt_Spike (with a 5:1 ratio of Leishmania cells to DCs). The ability of the leishmaniae to be internalised by the DCs was confirmed and the presence of the spike protein as well inside the DCs was verified. The immunofluorescence assays were conducted using an anti-spike monoclonal antibody (FIG. 4).

6. In Vitro Experiments on Dendritic Cells: Expression of Cytokines and Surface Markers

The assays on DCs were completed. In particular, the DCs were differentiated starting from monocytes from the blood of ten healthy volunteer donors. The dendritic cells were then exposed to L. tarentolae Lt_WT and Lt_Spike (with a 5:1 ratio of Leishmania cells to DCs). The response of the DCs was subsequently determined in terms of:
cytokines released in the supernatant (IL-1β, IL-1 RA, IL-2, IL-4, IL-5, IL-6, IL-7, IL-8, IL-9, IL-10, IL-12 (p70), IL-13, IL-15, IL-17, EOTAXIN, FGF basic, G-CSF, GM-CSF, IFN-γ, IP-10, MCP-1, MIP-1a, PDGF-bb, MIP-1β, RANTES, TNF-α, VEGF);
surface markers, by cytofluorimetry (CD80, CD83, HLA-DRII, DC-SIGN);
gene expression of cytokines, surface markers and transcription factors (CD14, CD209, CD40, CD80, CD83, CD86, HLA-DRB1, IL1B, IL2, IL6, IL10, IL12A, IL13, IL15, IFN Y, TNF, STAT1, STAT4, STAT6, GATA3, TBX21).

Moreover, the actual internalisation of Leishmania in the DCs was further confirmed by microscopic observation.

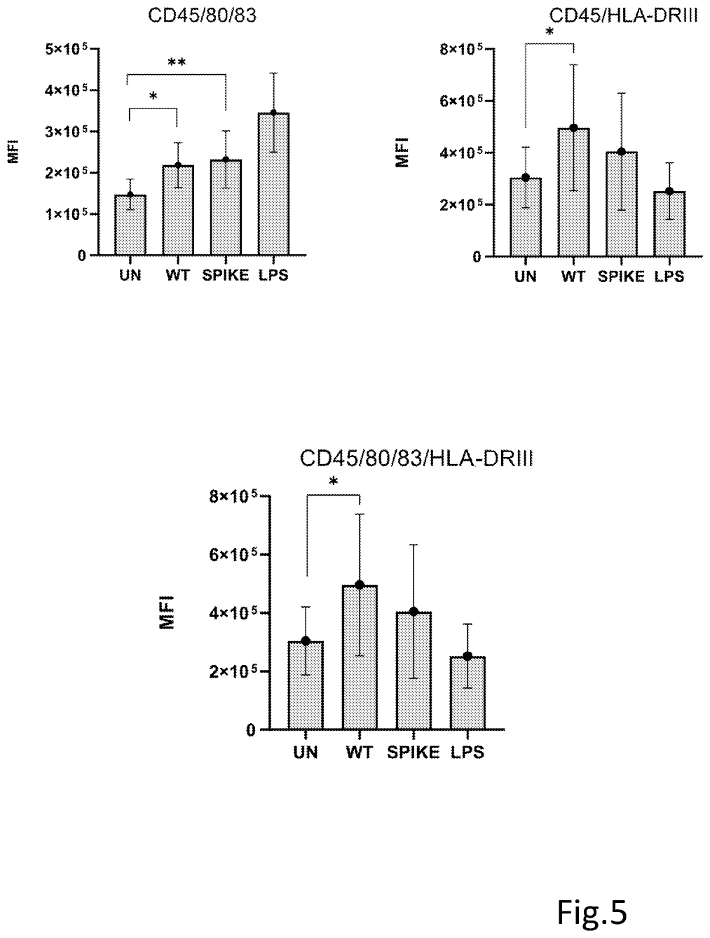

In summary, the results of these assays are the following:
The leishmaniae of both strains are internalised by the DCs;
The DCs respond to exposure to Leishmania with the expression of surface markers, indicative of an activation conducive to presentation of the antigen (CD80, CD83, HLA-DRII) (FIG. 5);
The variations in cytokine expression determined by Lt_Spike do not generally show to be significant compared to Lt_WT; neither strain determines significant variations in the expression of cytokines, either of the Th1 or Th2 type, indicating that Leishmania tarentolae is a neutral vaccine vehicle, certainly not a pro-inflammatory one.

7. Assays on a Murine Model

7.1. Determination of the Doses to be Administered

In a first assay on a murine model (female BALB/c mice), different doses and combinations in the administration of the Lt_Spike and RBD-SD1 antigens were compared. In particular, the Lt_Spike antigen (consisting of spike-expressing cells of Leishmania) was administered in inactivated form. The experiments were carried out on a total of 14 groups of animals (for a total of 70 animals); different doses and combinations of the two antigens and different routes of administration were tested. Altogether, three doses of the antigen were administered (at days 0, 21 and 47). Blood samples were taken at intermediate times (21 and 47), on a limited number of animals, to determine whether there was a response prior to administration of the third dose. At the end of the experiment (day 62), the sera were collected and the antibody response against the spike protein of the SARS-COV-2 virus was determined by ELISA.

The results obtained indicate that:
None of the antigens administered, in the different types of doses, combinations and routes of administration, caused any pathology detectable in the animal. Furthermore, no weight losses were observed; in fact, the treated animals, about eight weeks old at the beginning of the assay, gained weight, to a degree comparable to what was observed in the control groups;
On the basis of the above, the highest dose of Lt-Spike (2×10⁷ per animal subcutaneously) can be considered as well tolerated by the animals and they can thus be used for subsequent experimental phases;
In general, the antibody response against the spike protein manifested itself only after administration of the third dose.

It should be noted that the above-described assay was performed without the use of adjuvants. It may be concluded that inactivated cells of Leishmania tarentolae (as in Lt-Spike) do not perform an effective immunostimulant role. It follows that administration in association with an adjuvant is necessary.

7.2. Subcutaneous and Enteral Administration with Adjuvants; Determination of IgA Response

As adjuvants the following were tested: AddaVax (oil-in-water squalene emulsion); Adju-Phos (aluminium phosphate); R848 (Resiquimod).

On the basis of the results described in paragraph 7.1, the most promising type of antigen for the purpose of determining an antibody response in a murine model was Lt_Spike at the highest dose (2×10⁷ inactivated cells) associated with purified RBD-SD1. Again in consideration of the results described in paragraph 7.1, three types of adjuvants were tested: AddaVax (a commercial squalene-based preparation, similar to MF59) and aluminium salts (Adju-Phos) for subcutaneous administration; Resiquimod (R848) for rectal administration. An experiment was then carried out on a larger number of animals per group. It is noted that rectal administration was performed in order to obtain a proof of principle as to the possibility of enteral administration: a positive outcome of rectal administration, in terms of a neutralising antibody response, would justify further research investments aimed at the development of a gastro-resistant preparation for oral administration. At the end of the experiment, the immune response in the animals was determined in terms of: antibody response by means of ELISA assays; neutralising response by means of serum neutralisation assays on the SARS-COV-2 virus; and response of the T cell compartment, by means of assays on splenocytes. Furthermore, the production of secretory IgA antibodies was determined in the faeces of the animals, collected prior to sacrifice.

Specifically, the second assay on the murine model was performed on 10 groups of ten animals each:

Subcutaneous Administration
Group 1: Lt_Spike+AddaVax+RBD_SD1
Group 2: Lt_Spike+Adju-Phos+RBD_SD1
Group 3: AddaVax+RBD_SD1
Group 4: Adju-Phos+RBD_SD1
Control Group

Rectal Administration
Group 5: Lt_Spike+R848 (10 ug)+RBD_SD1
Group 6: Lt_Spike+R848 (25 ug)+RBD_SD1
Group 7: Lt_Spike+RBD_SD1
Group 8: RBD_SD1+R848
Control Group

In this experiment, as in the one described in paragraph 7.1, no detectable effects were observed in the animals, which, on the contrary, showed an increase in weight over the weeks of the study, in a manner comparable to what was observed in the control animals. Furthermore, part of the animals, after sacrifice, were subjected to thorough anatomical and histopathological observations, which did not reveal organ alterations in any of the treated animals.

The results obtained indicated that the adjuvant AddaVax was the most effective in bringing about a neutralising antibody response after subcutaneous administration. In rectal administration, the combined antigen (Lt_Spike+RBD-SD1) determined a specific IgG antibody response in 14 animals out of a total of 20 treated; a neutralising response was observed in 7 animals, whereas the presence of IgA was found in the faeces of 9 animals.

It should be stressed that the results obtained after rectal administration, albeit with a neutralising response in only 7 animals, are to be considered as very promising, for the following reason. Rectal administration shows elements of uncertainty, as it is impossible to control defecation of the animals after rectal administration of the vaccine. In fact, it was observed that some animals defecated immediately after administration. It is thus possible that d in the minutes/hours following administration. Therefore, it is believed that the results obtained are absolutely encouraging for the purpose of developing a preparation for oral administration, with capsules or pills with gastroprotection produced for a controlled release at the level of the ileum-colon. In fact, whereas rectal administration implies the risk of a rapid and uncontrollable expulsion of the antigen by defecation, it is assumed that oral administration with gastroprotection may ensure that the antigen will remain longer at the intestinal site, with less variability among the different animals as regards the dwell time (and hence the absorption time) of the antigen at the intestinal site.

8. Production of the Antigen in the Absence of Antibiotic Pressure; Scalability of Production

For the purpose of scalability, at an industrial level, of both Lt_Spike and Leishmania secreting RBD-SD1, it is necessary to maintain production of the recombined proteins in the absence of antibiotic pressure, in order to ensure the possibility of production, in industrial bioreactors, by strains from which the resistance gene for the selection of the transformed strains has been removed.

The cultures were maintained for about 8 weeks, with passages every 3-4 days (twice a week). At regular weekly intervals, the expression of the protein was verified by western blot (WB) analysis using an antibody specific for detection of the protein. To date (experiment still ongoing) the production of the protein, in the presence and absence of nourseothricin, was verified at generations 5, 20, 30, 40, 45, 50 and 100. The results indicate that the protein is produced without significant variations after 100 generations in culture, without the addition of the selection antibiotic, nourseothricin.

9. Lyophilisation of Antigens and Preparation of Formulations for Oral Administration

For the purpose of producing a formulation for oral administration in a murine model, a protocol was developed for the lyophilisation of the two antigens (Lt_Spike and RBD-SD1) associated with the adjuvant R848. Activities are currently underway for the production of gastro-protected, controlled-release capsules for the release of the antigen in the terminal portion of the ileum, the objective being the acquisition of the antigen by intestinal lymphoid tissue at the level of the ileum and colon. The selected murine model is the rat, in relation to the size of the capsules.

1. A vaccine composition comprising a host cell belonging to the genus Leishmania, said host cell comprising a polynucleotide coding for at least one protein of a virus belonging to the family Coronaviridae or for at least one portion of said protein.

2. The composition according to claim 1, wherein the at least one protein is a spike glycoprotein.

3. The composition according to claim 2, wherein the at least one protein is a spike glycoprotein, of a coronavirus selected from: SARS-COV, MERS-COV and SARS-COV-2.

4. The composition according to claim 1, wherein the polynucleotide comprises a sequence that is at least 80% identical to SEQ ID NO: 1 or SEQ ID NO.2.

5. The composition according to claim 4, wherein the polynucleotide comprises a sequence that is at least 90% identical to SEQ ID NO:1 or SEQ ID NO.2.

6. The composition according to claim 4, wherein the polynucleotide comprises a sequence that consists in SEQ ID NO: 1 or SEQ ID NO.2.

7. The composition according to claim 1, wherein the at least one protein comprises an amino acid sequence that is at least 80% identical to SEQ ID NO: 3 or SEQ ID NO.4.

8. The composition according to claim 7, wherein the at least one protein comprises an amino acid sequence that consists in SEQ ID NO:3 or SEQ ID NO.4.

9. (canceled)

10. The composition according to claim 1, wherein the host cell belongs to a species of Leishmania selected from: L. tarentolae, L. infantum. L. donovani, and L. major.

11. The composition according to claim 1, formulated as a solution, suspension, sterile emulsion or powder to be resuspended prior to use, or it is formulated as pills, capsules, tablets, granular powder, hard-shelled capsules, orally dissolving granules, sachets, lozenges or drinkable vials, or it is formulated for administration by inhalation.

12. (canceled)

13. A method for preventing a viral infection or a pathology caused by a virus belonging to the family Coronaviridae, said method comprising at least one step of administering to an individual in need thereof a vaccine composition according to claim 1.

14. The method according to claim 13, wherein the virus is selected from: SARS-COV, MERS-COV and SARS-COV-2.

15. The method according to claim 14, wherein the virus is SARS-COV-2.

16. The method according to claim 15, wherein the vaccine composition is administered in association or in combination with at least one protein comprising an amino acid sequence that is at least 80% identical to SEQ ID NO: 3 or SEQ ID NO.4.

17. A host cell belonging to the genus Leishmania comprising a polynucleotide coding for at least one protein of a virus belonging to the family Coronaviridae or for at least one portion of said protein, wherein the polynucleotide is according to claim 4.

18. A process for preparing a vaccine composition, comprising the steps of:
a) introducing, into a host cell of Leishmania, a vector, comprising a polynucleotide coding for at least one protein of a virus belonging to the family Coronaviridae or for at least one portion of said protein;
(b) selecting at least one host cell produced in step (a) that expresses said protein or portion of said protein; and
(c) suspending the at least one cell selected in step (b) with pharmaceutically acceptable salts/excipients.

19. The process according to claim 18, wherein the polynucleotide comprises a sequence that is at least 80% identical to SEQ ID NO:1 or SEQ ID NO.2.

20. The composition according to claim 3, wherein the spike glycoprotein is of SARS-COV-2.

21. The composition according to claim 1, wherein the at least one protein comprises an amino acid sequence that is at least 90% identical to SEQ ID NO: 3 or SEQ ID NO.4.

22. The composition according to claim 9, wherein the host cell belongs to the species L. tarentolae,

23. The method according to claim 16, wherein the at least one protein comprises an amino acid sequence that is at least 90% identical to SEQ ID NO: 3 or SEQ ID NO.4.


SEQUENCE LISTING (4 sequences)

SEQ ID NO: 1 - DNA, 3720 bp, Artificial Sequence
  Coding sequence for Sars Cov2 Spike
gttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggttctgcatctagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaa

SEQ ID NO: 2 - DNA, 825 bp, Artificial Sequence
  Sequence codifiyng for Sars-CoV-2  RBD-SD1
atggcccgtgtgcagccaactgagagcattgtgcgcttcccgaacatcacgaacctgtgcccattcggcgaggtgttcaacgcgacacgtttcgcgagcgtgtacgcgtggaaccgcaagcgcattagcaactgcgtggcggactacagcgtgctgtacaacagcgcgagcttcagcacgttcaagtgctacggcgtgtcgccgacgaagctgaacgacctgtgcttcacgaacgtgtacgccgacagctttgtgatccgtggcgacgaggtccgccagattgcaccaggccagacaggcaagatcgccgactacaactacaagctgccggacgactttacgggctgcgtgatcgcatggaacagcaacaacctggacagcaaggtcggcggcaactacaactacctgtaccgcctgttccgcaagagcaacctgaagccgtttgagcgcgacatcagcaccgagatctaccaggcaggttctacgccatgcaacggcgtcgagggcttcaactgctactttccgctgcagtcgtacggcttccagccgacaaacggtgtcggctaccagccataccgtgtggtggtgctgtctttcgagctgctgcacgcaccagcaacagtgtgcggtccaaagaagtccaccaacctggtcaagaacaagtgcgtgaacttcaacttcaacggcctgaccggcacgggcgtgctgacagagagcaacaagaagttcctgccgttccagcagttcggccgcgacattgcagacacgacagatgcagtgcgcgacccgcagacgcttgagatcctggacattacaccgtgcagc

SEQ ID NO: 3 - protein, 1523 aa, Artificial Sequence
  Amino acid sequence of SARS-CoV-2 Spike
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKGTHHHHHHMARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 4 - protein, 275 aa, Artificial Sequence
  Amino acid sequence of RBD-SD1 of SARS-CoV-2
MARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS